(19) United States
(12) Patent Application Publication
(10) Pub. No.: US 2025/0018124 A1
(43) Pub. Date: Jan. 16, 2025

(54) USER EQUIPMENT, ELECTRONIC SYSTEM, DRUG DELIVERY DEVICE, METHOD, MACHINE-READABLE CODE, DATA STORAGE MEDIUM, AND SET

(71) Applicant: Sanofi, Paris (FR)

(72) Inventors: Stefan Alt, Frankfurt am Main (DE); Michael Helmer, Frankfurt am Main (DE)

(21) Appl. No.: 18/705,856

(22) PCT Filed: Nov. 1, 2022
(86) PCT No.: PCT/EP2022/080406

§ 371 Date: Apr. 29, 2024

(30) Foreign Application Priority Data
Nov. 3, 2021 (EP) 21315206.9

(51) Int. Cl. A61M 5/315

(52) U.S. Cl. CPC A61M 5/31546; A61M 2205/502

(57) ABSTRACT

A user equipment is proposed comprising an electronic control unit (42) and a wireless communication unit (44) operatively connected to the electronic control unit, the electronic control unit (42) being configured to control operation of the wireless communication unit (44), the wireless communication unit being operable to determine at least one characteristic associated with at least one component of a drug delivery device (10). Furthermore, an electronic system, a drug delivery device, a method, a machine-readable code, a data storage medium, and a set are provided.

### BACKGROUND

The present disclosure relates to a user equipment, preferably a user equipment for controlling operation of a drug delivery device or for preparing a drug delivery device for operation, such as for a dosing operation. The disclosure further relates to an electronic system for a drug delivery device, one or more components for the drug delivery device, to a drug delivery device, and a method for preparing the drug delivery device for operation. Further, machine-readable code, a data storage medium, and a set are provided.

Various types of pen-type and needle-based injection devices are known. Needle-based injection system (NIS) devices are used by patients to conveniently perform frequent (e.g. sub-cutaneous) injections of drugs, e.g. insulin for diabetics. These NIS devices usually have a mechanism to select or set the dose of the drug to be injected within certain limits, i.e. the dose to be injected may be varied within these limits. Recently NIS devices tend to include more and more electronics to measure and/or detect the selected and/or injected doses, and subsequently store it in an internal memory and/or transmit it to an external device such as for the purpose of dose logging.

Some users or patients may need to administer different drugs using different devices, where the devices may, at first glance, look similar. Also, devices may be kept in households where a variety of people are in theory able to access the devices.

### SUMMARY

It is an object of the present disclosure to provide improvements, particularly improvements related to drug delivery devices or equipment, or systems intended for operating together with or integrated into drug delivery devices.

This object is achieved by the subject-matter of the independent claims. Advantageous embodiments and refinements are subject-matter of the dependent claims. We note that this disclosure aside from the solutions claimed in the independent claims may provide other advantageous solutions or features.

One aspect of the present disclosure relates to a user equipment.

In one embodiment, the user equipment comprises an electronic control unit. The user equipment further comprises a wireless communication unit operatively connected to the electronic control unit. The electronic control unit is configured to control operation of the wireless communication unit. The wireless communication unit is operable to determine at least one characteristic associated with at least one component of a drug delivery device. The electronic control unit is preferably configured to evaluate the at least one determined characteristic.

In one embodiment, based on the result of the evaluation or the determined characteristic, the user equipment may be configured to communicate with the drug delivery device, e.g. to change the state of the device, or with the user, e.g. to indicate whether the device with which the equipment is communicating is a device the user should use for an upcoming drug administration or not.

In one embodiment, based on the result of the evaluation of the at least one determined characteristic, the electronic control unit is configured to issue or to decide whether to issue one or more control unit commands. The control unit command may be a command to influence the state of the user equipment, e.g. to cause that information is provided to the user by the equipment, or to influence the state of the drug delivery device, e.g. via the wireless communication unit.

In one embodiment, the user equipment comprises an equipment signaling unit.

In one embodiment, the one or more control unit commands comprise a command configured to cause the equipment signaling unit of the user equipment to present information relating to the drug delivery device to the user. The indicated information may be based on the at least one determined characteristic or the result of the evaluation of the characteristic.

In one embodiment, the one or more control unit commands comprise a command configured to cause the wireless communication unit to send a system command to an electronic system of or for the drug delivery device. Via the system command the state of the device may be changed.

In one embodiment, the electronic control unit is configured to decide, e.g. based on the result of the evaluation, whether the wireless communication unit should send at least one unlock command as the system command to the electronic system. The unlock command may be sent to unlock or activate at least one usability function of the drug delivery device.

In one embodiment, the user equipment is configured to issue or send the control unit command (e.g. the system command or the command sent to the equipment signaling unit) and/or the unlock command or system command when, preferably only when, the result of the evaluation is that the at least one determined characteristic meets at least one predetermined criterion, e.g. whether the device is within a predetermined distance from the user equipment and/or whether components of the device are compatible with each other. The latter is particularly expedient when the components are releasably connected with each other, such as a container module for holding a medicament and a mechanism module which comprises a drive mechanism operable to set a dose in a dose setting operation and delivering the set dose in a dose delivery operation. Often one mechanism module can be used with a plurality of container modules with the same medicament when medicament provided by one container module has been delivered and the new container module is connected to the mechanism module. Having the device within a predetermined distance, facilitates to ensure that the user is handling the correct device.

In one embodiment, having a usability function unlocked (e.g. unlockable only) by the user equipment adds considerably to the safety. For example, without the user equipment-controlled unlocking of the usability function(s), the usability function, e.g. a dosing operation, may not be performed. The usability function may be unlocked if, preferably only if, the device is in the hand of the user or closer to the wireless communication unit than the hand of the user and/or if, preferably only if, the evaluation of the determined characteristic by the user equipment results in that the device should indeed be unlocked, e.g. as its components are compatible or as it is the correct device. This may include a determination whether the medicament which the device should administer is the one which the user currently needs or should administer according to a medicament administration scheme.

In one embodiment, the equipment signaling unit is configured to indicate to the user whether the predetermined criterion is met. Signaling to the user whether the components are compatible or incompatible and/or whether the device is within a predetermined distance from the user equipment is advantageous and increases user safety.

In one embodiment, the user equipment comprises a memory, e.g. a non-volatile memory. The memory may be configured to store dose history information on the medicament amounts or dose amounts delivered in dose delivery operations performed by the user. The memory may store information about the medicament which the user needs to administer. The user equipment may comprise a reminder functionality, which is operable to remind the user, e.g. visually or audible, when there is a need to administer medicament, e.g. by comparing time information relating to the last administration of a particular medicament with the current time or an administration schedule. The memory may store pairing information on with which drug delivery device the user equipment is allowed to communicate.

In one embodiment, the user equipment is an electronic device, preferably for personal use. The user equipment may be a device separate from the drug delivery device. The user equipment may be expected to have a product live time longer than that of the drug delivery device. The user equipment may be or comprise a general purpose computation unit. This unit may serve as the electronic control unit.

In one embodiment, the user equipment is a wearable device. Wearable devices are devices which are worn by the user in a way such that the device is connected to the body of the user during its intended use and, preferably, always is at the same location relative to the body or within the same body region during the intended use. The wearable device may be a wrist-worn device or wrist-wearable device. The wrist is close to the user's hand and, hence, communication with a drug delivery device or the elements thereof which is held by the user in the hand close to the wrist or when the device is being brought close to the user equipment is facilitated, e.g. if near-field communication is used as wireless communication technology. The wearable device may be a smart watch or smart bracelet, for example. As an alternative to a wearable device, the user equipment may be a handheld device, e.g. a smartphone or tablet PC. As a further alternative, the user equipment may be a dedicated purpose device, specifically for controlling and/or managing one or more drug delivery devices—e.g. it might be a bracelet or ID-badge or the like worn by authorized personnel of a medical facility or nursing home.

In one embodiment, the electronic control unit comprises a processor unit and/or an ASIC (application specific integrated circuit). The electronic control unit may comprise memory for storing data and/or for storing machine-readable code that is executed in the processor unit. The electronic control unit may be electrically conductively connected to the wireless communication unit, e.g. via conductors on a circuit board.

In one embodiment, the wireless communication unit is or comprises an electronic circuit configured to send and/or receive waves, e.g. radio waves, such as to establish a data transfer connection. The unlock command may be sent via a different communication circuit than the one which was used for determining the characteristic. The wireless communication unit may comprise at least one wireless communication circuit that transceives (transmits and/or receives) in a specific frequency range and/or that follows a specific transmission protocol. The wireless communication unit may comprise multiple communication circuits, each transceiving in a different frequency range and/or according to a different wireless transmission protocol. The wireless communication unit may comprise at least one RFID-communication circuit (radio frequency identification). The wireless communication unit may comprise an NFC-circuit (near-field communication), e.g. an active NFC-circuit (preferably with associated power source, e.g. a battery such as a printable battery) or a passive NFC-circuit (preferably without a power source powering the circuit). The wireless communication unit may comprise at least one Bluetooth and/or BLE communication circuit (Bluetooth Low Energy). The wireless communication unit may comprise at least one WiFi-communication circuit (wireless fidelity, e.g. according to the IEEE 802.11 standard/protocol). Alternatively or additionally, the wireless communication unit may comprise a magnetometer/compass circuit to detect variation in magnetic fields, e.g. variations produced by a wireless communication circuit of a component of the drug delivery device.

In one embodiment, the “wireless communication unit being operable to determine” may refer to the reception of and/or the evaluation of at least one signal by the wireless communication unit. The signal may be received in response (or as a response) to a request sent via the wireless communication unit, e.g. to the component of the drug delivery device.

In one embodiment, the drug delivery device is a device configured to eject a medicament from a container and provide it to a patient. The drug delivery device may comprise a needle or be provided to be connected with a needle to transfer the medicament from the container to the patient, e.g. when the needle is inserted into the patient's body. The drug delivery device may comprise a number of components (e.g. modules such as pre-assembled modules). The components of the drug delivery device may comprise an electronic system (electronics module), a drive mechanism (mechanism module), a medicament container and/or a container holder for the medicament container (container module). The container may be a cartridge. The holder may be configured to receive the container. The electronics module and the mechanism module may be integrated into a common module or the electronics module may be, e.g. releasably, attachable to the mechanism module. The container module and the mechanism module may be permanently or releasably connected or connectable.

In one embodiment, the drive mechanism is a mechanism configured to force the medicament from its container. The drive mechanism may include a converter unit to convert energy provided by the user and/or an energy storage, e.g. at least one mechanical spring, an electronic battery (one or more chemical cells) or pressurized air or other suitable means, into a motion that forces the medicament to leave the container. The drive mechanism may comprise a piston rod that is moved axially to force the medicament from the container, e.g. by displacing a plunger or bung of the container towards a dispensing end of the container.

In one embodiment, the usability function of the drug delivery device is a function that is essential for a proper use of the drug delivery device. Preferably, without the usability function being performable, the device cannot be operated. When the usability function is locked, e.g. before it is unlocked, the functionality may not be performed or the drug delivery device may not be used.

In one embodiment, the at least one characteristic comprises the distance between or the proximity of the component and the user equipment, e.g. its wireless communication unit. Evaluating the at least one determined characteristic may comprise determining whether the component or the entire drug delivery device is within a predetermined distance of the user equipment. When, preferably only when, the result of the evaluation is that the component or the drug delivery device is within the predetermined distance, the unlock command is sent or the electronic control unit instructs sending of the unlock command, e.g. via the wireless communication unit. Thus, proximity of user equipment and component or device may be one criterion which has to be met for the unlock command to be sent. The evaluation may comprise the determination or monitoring of the distance or checking whether a signal is received from a near-field communication circuit of the device, which already implies that there is proximity due to the short range of the near-field communication.

The at least one determined characteristic may comprise the distance and/or proximity of the component, distances and/or proximity of at least two of the components or distances and/or proximity of all of the components. The electronic control unit may be configured to send the unlock command when, e.g. only when, the at least one component, e.g. at least two of the components, e.g. all components, of the drug delivery device is determined to be within the predetermined distance of the user equipment. The component may be evaluated to be in proximity when a response signal is received after a request signal was sent (e.g. when very short-range communication modules are used like NFC) and/or when the distance (e.g. determined via signal strength detection/evaluation (RSSI) and/or time of flight measurement), is less than 2 m, e.g. less than 1 m, e.g. less than 0.5 m, less than 20 cm, less than 15 cm, less than 10 cm. This may ensure that the drug delivery device is only used in proximity of the intended user and not by unauthorized users. Preferably the predetermined distance is less than or equal to the distance between the wrist and the foremost fingertip when the hand is flat or than or equal to the distance between the wrist and the end of a first remote from the wrist when the hand adjoining the wrist, preferably that wrist on which the user equipment is or is to be worn, forms the fist.

In one embodiment, the predetermined distance is less than or equal to one of the following values: 20 cm, 15 cm, 10 cm, 9 cm, 8 cm, 7 cm, 6 cm, 5 cm, 4 cm, 3 cm, 2 cm, 1 cm, 5 mm, 3 mm, 2 mm, 1.5 mm, 1 mm.

In one embodiment, the user equipment is configured to determine whether the drug delivery device or its component(s) is within a predetermined distance from the user equipment which is typical for the distance between a wrist-worn device and the user's hand or the palm of the user's hand, preferably the hand associated with the wrist, e.g. 7 to 14 cm. Hence, the operation of the drug delivery device may be controlled by the user equipment if it is at least likely that the user holds the device in his hand. Only when it has been confirmed that the user likely holds the device in his hand, the unlock command may be sent. For this purpose, technologies different from NFC may be required for distance measurement and/or monitoring (see above), especially if the hand is the one adjoining the wrist on the same arm. In case the other hand is used to bring the device close to the (wrist-worn) user equipment, near field communication may be employed as well.

In one embodiment, in order to be evaluated to be within the predetermined distance, the drug delivery device or its component(s) have to be brought into closer proximity to the wrist-worn device than the distance to the hand adjoining the wrist on the same arm, e.g. when using NFC circuits. This, however, also requires the user to get into closer proximity to a particular device.

In one embodiment, the at least one determined characteristic comprises identifiers of at least two components of the drug delivery device. The respective identifier may be contained in or provided by an identifier circuit. Evaluating the at least one determined characteristic may comprise determining whether the at least two components of the drug delivery device are compatible with one another. The user equipment, e.g. the electronic control unit, may be configured to send the unlock command when, preferably only when, the components are compatible with one another, expediently after the evaluation of the characteristic by the electronic control unit. Thus, compatibility of drug delivery device components may be a criterion which has to be met for the unlock command to be sent. The identifiers may be or may comprise component-type identifiers, medicament-type identifiers, and/or device identifiers. The identifiers may be or may comprise product IDs. The electronic control unit may be configured to compare the determined identifiers of the components with a look-up table stored in memory to determine or evaluate the compatibility. The drug delivery device, especially the drive mechanism, may be only compatible with or designed for specific medicaments or medicament formulations. Hence, the evaluation whether the components are compatible may comprise a check whether the drive mechanism is designed for the container module or for delivering the medicament contained in the container module. The drive mechanism may be configured such that for a pre-defined dose setting operation, e.g. a rotation of a dose setting member by a defined angle, the axial displacement of the piston rod, which usually correlates with the amount of medicament ejected when the same container is used, is always the same regardless of the medicament which is used with the drive mechanism. Hence, if a medicament is administered with a drive mechanism which the mechanism has not been designed for, there is a severe risk of applying the wrong dose, either an overdose or an underdose, e.g. because the concentrations of an active ingredient in the medicament or the container dimensions may be different for the correct medicament and the wrong medicament. Also, different drive mechanism may be designed for different medicament volumes, e.g. with respect to a dose setting limiting functionality drug delivery devices often have. This functionality prevents the setting of a dose which exceeds the amount of liquid remaining in the container. Hence, the evaluation may comprise a check, whether the mechanism and the medicament or the medicament container are suitable for being used together. Unlocking the usability function only when the components (modules) match or are compatible with one another may reduce the risk of the wrong medicament and/or wrong medicament amount being applied with a particular drive mechanism or vice versa.

In one embodiment, the electronic control unit is configured to send the unlock command only when proximity as well as compatibility has been confirmed. However, one check regarding proximity or compatibility for triggering the unlock command is also covered by the present disclosure.

In one embodiment, the user equipment is configured to determine a first identifier of a first component and a second identifier of a second component of the device in one relative position with respect to the drug delivery device, e.g. simultaneously. Thus, the first identifier may be situated on the first component and the second identifier may be situated on the second component. Both identifiers may be in the communication range of the wireless communication unit, e.g. one communication circuit of the unit, such as an NFC circuit. The first and/or second identifier may be or may comprise a passive or active NFC circuit. The relative position may be close to the wireless communication unit, e.g. within 2 cm or less or 1 cm or less or 0.5 cm or less of the unit, e.g. within 1 mm of the unit.

In one embodiment, the user equipment comprises the equipment signaling unit, wherein the equipment signaling unit is preferably operatively connected to the electronic control unit. The signaling unit may be formed by or comprise a display. The signaling unit may be configured to provide audible and/or visual indications, e.g. via a speaker or a multi-pixel display. The signaling unit may be configured to indicate, preferably upon instruction by the electronic control unit, at least one of, an arbitrarily selected plurality of or all of the following, e.g. to the user:
an alarm for a due delivery operation or medicament administration, e.g. an audible and/or visible alarm signal; The determination whether the administration is due can be made by the electronic control unit based on values of the dose history or administration history retrieved from the drug delivery device or from an internal memory of the user equipment; and/or
a device identification to indicate a drug delivery device which the user should use for an upcoming medicament administration in a dose delivery operation; The indication of the device identification may be effected before the user selects the device for the delivery operation. The indication of the device identification on the signaling unit may be correlated with an indication given on the device, e.g. a mark of the same color, the same symbol or the same text. The indication given on the device may be permanent, e.g. a color mark, or be provided by a device signaling unit, e.g. upon receipt of a request by the user equipment. The indication of the device identification may be a feature differentiating the drug delivery device to be used from other, e.g. visually similar, devices. The electronic control unit may be configured to determine the device identification after a pairing process with the drug delivery device, e.g. from a device ID or by user-entry. Alternatively or additionally, the device identification may be a characteristic, e.g. a visible and/or audible signal, only activated by the drug delivery device temporarily during an alarm state for a due medicament administration, e.g. a blinking light (LED), such as of a specific color or a symbol, e.g. one that is only visible when backlit. This may prevent mix-ups of similar devices used in the same household; and/or
a ready state of the drug delivery device, e.g. after having sent the unlock command; The device may have a locked state, e.g. a state where a dosing operation, e.g. a dose setting operation and/or a dose delivery operation, is not possible, and the ready state, e.g. a state in which the dosing operation is possible. The ready state may be indicated or the drug delivery device may be in the ready state when, e.g. only when, the device has been unlocked and the dosing operation has become possible; and/or
information on whether the criterion, in respect of which the at least one determined characteristic is evaluated, is met (or not). In other words, the signaling unit may indicate whether the components of the device are compatible and/or whether the distance between the user equipment and the device is small enough. Hence, the signaling unit may indicate information on the compatibility of components of the drug delivery device, e.g. components which are releasably connectable to one another, e.g. a mechanism module and a container module. If the components are found to be incompatible, the user may be prompted to substitute one of the components with another one by the information indicated by the signaling unit. If compatibility is confirmed, the indicated information may be reassuring for the user that the device is properly set up. If the device is found to not meet the distance or proximity criterion, the user may be prompted to use a different device via the signaling unit; and/or
a dose amount to be set in the drug delivery device for the dose delivery operation, preferably after indicating the ready state and/or after the compatibility of the components of the device and/or proximity has been confirmed. The dose amount may be calculated by the user equipment or retrieved from an electronic system of the drug delivery device. The calculation of the dose amount may involve consideration of the time passed since the latest medicament administration, i.e. the latest drug delivery operation, the dose amount delivered in that administration, and/or a time until a next planned/scheduled administration. The electronic control unit may be configured to adapt the dose amount to be set dependent on a component-type identifier of the medicament container, cartridge holder and/or drive mechanism. The dose amount to be set may depend on the combination of components used, e.g. to compensate for different parameters like container diameter or medicament. However, it is also possible, that the drive mechanism is just applicable for one medicament formulation in a specific container with a specific volume of liquid medicament in it.

Another aspect of the disclosure relates to an electronic system for a drug delivery device.

In one embodiment, the electronic system comprises a system wireless communication unit. The system wireless communication unit may be configured to communicate with the user equipment, preferably with the wireless communication unit of the user equipment, e.g. via a different circuit than the one used for proximity confirmation. The system wireless communication unit is preferably configured to wirelessly communicate with, e.g. to receive an unlock command from, a user equipment, e.g. the user equipment as described further above. The electronic system further comprises a system electronic control unit. The system electronic control unit is operatively connected to the system wireless communication unit. The system electronic control unit may be configured to, in response to the unlock command, issue an activation command to activate at least one usability function of the drug delivery device. The electronic system may be integrated into the mechanism module or, e.g. releasably, coupleable or coupled to that module. Communication unit and control unit may be, in general, configured as described further above for the control unit of the user equipment. However, for the electronic system, an ASIC may be particularly suitable.

In one embodiment, activating or unlocking the at least one usability function comprises:
switching on or revealing a dose display or indication member of the drug delivery device. The dose display may be configured to indicate the currently set dose amount to the user, e.g. through a window, preferably in the housing of the device or of the drive mechanism. During the dose setting operation the dose amount may be increased or decreased. The set dose amount may be delivered in the dose delivery operation. The dose display may be an electronically or mechanically operated display. A mechanically operated display may comprise an indication member visible through and/or movable relative to the window to change the indicated dose amount in the window. In the locked state of the device, e.g. before the unlock command has been sent, the indication member or indicia on that member may be not visible through the window. In the ready state the indication member may be visible through the window. In the locked state of the device, a shutter member may be arranged between the indication member and the window. Thus, the user cannot view the indication member in the locked state. The shutter member may be removed to reveal the indication member and, hence, the dose display, e.g. in response to the unlock command. The indication member may be a sleeve, e.g. a number sleeve. The indication member may be configured to move, e.g. rotate, such as in helical fashion, such that a number proportional to the set dose is viewable/readable in the window during dose setting. Alternatively to having a shutter member, before the unlock command is received, the string of numbers may be misaligned in relation to the window such that the numbers are not viewable or readable through the window. The dose display may be revealed by shifting the number sleeve and/or the window into a position such that the string of numbers aligns with the window.
enabling a dosing operation of the drug delivery device, e.g. unlocking a dose member of the drug delivery device for a manipulation of the dose member to perform a dose setting operation or a dose delivery operation. The dosing operation which is enabled may be the dose setting operation or the dose delivery operation. In the locked state, e.g. before the unlock command has been sent, the dosing operation may be prevented. In the ready state, after having received the unlock command the dosing operation is possible. For example, a dose member which is arranged to be touched by the user for dose setting may be not movable, e.g. axially and/or rotationally locked relative to the housing, and/or the dose member may be decoupled from the drive mechanism, e.g. of the mechanism module, such that a dose setting operation cannot be performed in the locked state. In the ready state, the dose setting operation is expediently possible. It is preferred, that the dose setting operation is prevented in the locked state.

In one embodiment, the user equipment and the electronic system are paired. Consequently, the electronic system may accept unlock commands only from a paired user equipment and not from any other user equipment. For pairing, a device ID code may have to be entered and stored in a, e.g. non-volatile, memory of the user equipment. Other pairing procedures are possible of course.

In one embodiment, activating or unlocking the at least one usability function comprises unlocking a dose member of the drug delivery device. When locked, the dose member may not be movable in a dose setting direction, e.g. relative to the housing. When the dose member is unlocked this movement may be possible. The dose member may be a dose setting member and/or a dose delivery activation member.

In one embodiment, in response to the activation command, the drug delivery device or the drive mechanism is configured such that the dose member is moved relative to a housing, e.g. of the mechanism module, from a locked position or non-operating position into an operating position. The operating position may be proximally offset from the locked or non-operating position. In the locked or non-operating position the dose member may be rotationally and/or axially locked to the housing. Thus, a rotation of the dose member, which may be necessary to set a dose, may be prevented. Alternatively or additionally, the dose member may be decoupled from a dose setting mechanism (e.g. from the drive mechanism) in the locked or non-operating position and coupled to the dose setting mechanism in the operating position. The movement into the operating position may be spring driven. In the locked position, a spring may be biased and may tend to move the dose member towards the operating position. The electronic control unit may be configured to unlock a mechanical lock in order to allow the (spring driven) movement into the operating position.

In one embodiment, the electronic system is integrated into the dose member. In other words, a housing of the electronic system may form the user interface surface of the device for the dosing operation.

In one embodiment, the system comprises a system signaling unit, e.g. an electronic display (e.g. liquid crystal or e-ink display) and/or an LED or a non-pixeled display. The system signaling unit may be configured, preferably upon a command by the system electronic control unit, to indicate an alarm regarding a drug delivery operation being due to the user.

In one embodiment, the system electronic control unit is configured to issue a ready command. The electronic control unit may be configured to cause the system signaling unit, in response to the ready command, to indicate to the user that the drug delivery device is ready, e.g. for commencing the dosing operation. Thus, the system signaling unit may indicate the ready state to the user. The system signaling unit may be the device signaling unit mentioned further above. The ready command may be issued only after the dosing operation has been enabled. Alternatively or additionally, the ready state may be indicated by the signaling unit of the user equipment. If the signaling units of the system and the equipment indicate the ready state, the indications on the different units are expediently correlated, e.g. using the same way of indication, such as the same color, flashing pattern of an LED or a display etc. The ready command may be generated after the activation or unlock command, e.g. time delayed, and/or in response to enabling of the dosing operation having been completed

Another aspect relates to a drug delivery device.

The device may be a needle based and/or pen-type device. The device may be a variable dose device, where the size of the dose of medicament delivered by the device may be set by the user. The device may be of the dial extension type, i.e. the length of the device may increase during the dose setting operation in an amount proportional to the size of the currently set dose. The device may be, partly or completely user driven. That is to say, during the dose delivery operation, the user may have to apply part of (e.g. in addition to a spring force) or all of the force required for the dose delivery operation. Alternatively, the device may be driven by an energy source, e.g. a spring. The energy source may be loaded by the user during the dose setting operation.

In one embodiment, the drug delivery device comprises one of, an arbitrarily selected plurality of or all of the following:
a container module as a component of the drug delivery device, the container module comprising or being configured to retain a container with medicament,
a mechanism module as a component of the drug delivery device, the mechanism module comprising a drive mechanism, e.g. for setting a dose of medicament to be delivered from the container and delivering the set dose (thus, the “drive mechanism” may encompass a “dose setting mechanism” and a “dose delivery mechanism”), and
an electronic system, e.g. as described further above, as a component of the drug delivery device or an interface for mechanical connection with such an electronic system, preferably in the mechanism module.

The container module and the mechanism module may be releasably connectable or connected with one another. For example, a proximal end of the container module may be connected with a distal end of the mechanism module, e.g. by a thread or bayonet connection.

The respective component of the delivery device may comprise an (component) identifier for being (wirelessly) communicated to and/or determined by the user equipment, preferably its wireless communication unit, described further above. The respective identifier may comprise or be provided by an active or passive NFC circuit.

In one embodiment, the identifier of the mechanism module and the identifier of the container module are in close proximity to one another, especially when the modules are connected. The identifiers may be arranged within 3 cm, within 2 cm, or within 1.5 cm from one another. The identifier on the mechanism module may be arranged in the distal end region of the mechanism module. The identifier on the container module may be arranged in the proximal end region of the container module. The identifier on the container module may be arranged distally relative to the identifier on the mechanism module. The respective identifier may comprise a wireless communication circuit, e.g. an active or passive NFC circuit.

In one embodiment, the device has a locked state, in which the dosing operation is not possible and a ready state in which the dosing operation is possible as described further above. The switching between the states may be governed by the user equipment and, expediently its unlock command being sent and received by the electronic system.

In one embodiment, the device is configured to return to the locked state after or when the dose delivery operation has been completed. For example, the dose member may be locked again relative to the housing, when it has reached an end position at the end of a dose delivery operation. The member may be moved distally relative to the housing into the end position from a dose set position. The lock may be mechanically or electronically operated or controlled.

Another aspect relates to a method for preparing a drug delivery device for a dosing operation or controlling the operation of the drug delivery device, e.g. a drug delivery device as described above, with the aid of a user equipment, e.g. a user equipment as described above, the method comprising the following steps:
wirelessly determining at least one characteristic of at least one component of the drug delivery device via the user equipment,
unlocking at least one usability function of the drug delivery device, e.g. when, preferably only when, the at least one determined characteristic meets a predefined criterion.

The user equipment may be configured to operate according to the method.

Another aspect relates to a machine-readable code that, when loaded on and/or executed by an electronic control unit of a user equipment, causes the user equipment to function as a user equipment as described further above or controls the method as described above.

Another aspect relates to a data storage medium, e.g. a physical data storage or a data stream, comprising the machine-readable code.

Another aspect relates to a set or an item. The set or item comprises one of, an arbitrarily selected plurality of or all of the following:
the user equipment as described above,
the electronic system as described above,
one or more components of the drug delivery device as described above,
the drug delivery device as described above,
the machine-readable code, as described above, and/or
the data storage medium, as described above.

We note that features described in conjunction with different embodiments or aspects may be combined with one another. We note that features described in conjunction with the entities user equipment, electronic system, drug delivery device, method, set, data storage medium, code above and below do also apply for other entities and do not only apply for the entity in the context of which they are disclosed. For example, features for the device do also relate to the method, the electronic system or the user equipment.

A member being “configured” to perform a specific task or function may refer to the member performing that task or function in at least one state of operation of the device. The member may be specially formed, programmed and/or arranged to allow for the performance of the task.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

Further features, advantages and expediencies of the present disclosure will become apparent from the following description of the exemplary embodiment in conjunction with the drawings.

### BRIEF DESCRIPTION OF THE DRAWINGS

FIG. 1 shows an embodiment of a drug delivery device in a locked state and a user equipment in a schematic view.

FIG. 2 shows the drug delivery device in an unlocked or ready state in a partial view.

### DESCRIPTION OF EXEMPLARY EMBODIMENTS

In the figures, identical elements, identically acting elements or elements of the same kind may be provided with the same reference numerals.

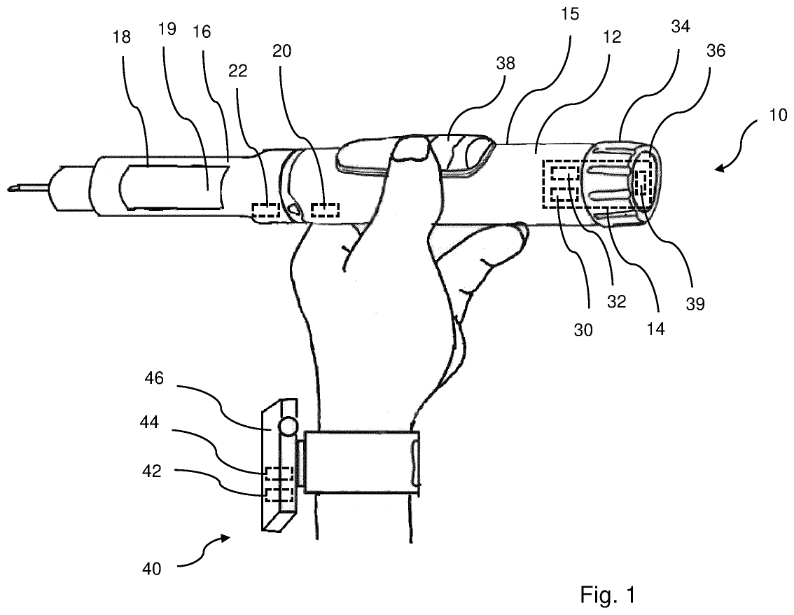

FIG. 1 shows a drug delivery device 10. The drug delivery device 10 is a needle-based drug delivery device. The drug delivery device 10 is a variable dose device. The drug delivery device 10 is an injection device. The drug delivery device 10 is a pen-type device, the form of which resembles a pen.

The drug delivery device 10 comprises two modules, e.g. a mechanism module and a container module. The drug delivery device 10 includes a drive mechanism 12 and an electronic system 14. The drive mechanism 12 (or dose setting and drive mechanism) and the electronic system 14 are combined into a first module, e.g. the mechanism module, in the depicted embodiment. That is to say, the electronic system 14 is integrated into the mechanism module. It is, however, also conceivable to have another module comprising the electronic system, which is (e.g. releasably) connectable to the mechanism module, e.g. as a button or user interface member which may provide electronic control functionality to the device. The drug delivery device 10 further comprises a container holder 16 (part of the second module). The holder 16 comprises a receptacle 18 for a medicament container 19. The holder 16 comprises the medicament container 19 (holder 16 and container 19 together may form the second module). The medicament container 19 comprises a liquid medicament. The amount of medicament in the container is expediently sufficient to deliver a plurality of user-settable doses, preferably even if the maximum dose deliverable with the device is set by the user. The medicament container 19 is preferably permanently retained in the holder 19. The medicament container may be a cartridge. A dispensing end of a cartridge body of the cartridge is covered by a pierceable septum (not explicitly shown). A proximal opening of the cartridge body is closed by a movable stopper or bung (not explicitly shown). If fluid communication between the cartridge body interior and the exterior is established, e.g. by a needle piercing the septum, and the stopper is moved distally towards a dispensing end of the cartridge (towards the septum) medicament will be delivered from the cartridge. The holder 16 comprises an interface to connect the cartridge holder with the first module. The housing 15 of the drive mechanism 12 (of the first module) includes an interface to connect to the holder 16. The interfaces of the cartridge holder 16 and the drive mechanism may for example comprises features for a threaded interface or a bayonet interface or the like to attach to each other. The housing 15 has a cylindrical shape.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys (B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

The drive mechanism 12 comprises a first wireless communication circuit 20. The first wireless communication circuit 20 is a passive NFC circuit (without power source) or an active NFC circuit, e.g. with a power source, such as a printable battery, where a passive NFC circuit may be more suitable, as it is more cost efficient. The holder 16 comprises a second wireless communication circuit 22. The second wireless communication circuit 22 is a passive NFC circuit or an active NFC circuit, e.g. with a printable battery, where a passive NFC circuit may be more suitable again. The first wireless communication circuit 20 is arranged near the interface of the first module, e.g. close to its distal end such as close to the distal end of housing 15. The second wireless communication circuit 22 is arranged near the interface of the second module, e.g. close to its proximal end. When the holder 16 is connected to the first module (the drive mechanism 12), the first and second wireless communication circuits 20, 22 are in close proximity to each other. In this way it is more likely for a user equipment 40 to be in reach of communication of both of the wireless communication circuits 20, 22 at the same time. The circuits 20 and 22 may be separated by less than 3 cm, e.g. 2 cm or less or 1.5 cm or less. According to alternative embodiments, a wireless communication circuit may be arranged on the medicament container 19 instead of or additionally to the wireless communication circuit 22 in the holder 16. This is particularly suitable, if the container can be removed from the holder and replaced with a new container. Otherwise, the assembly of container and container holder, which may be irreleasably secured to one another, may form one disposable item. In this case, either the container or the holder may be provided with the circuit.

FIG. 1 further shows a user equipment 40. The user equipment 40 is a smart watch in the depicted embodiment. A smart bracelet is a possible alternative embodiment or a handheld device such as a smartphone. Wearable devices are particularly suitable as user equipment in the context of the disclosure. The user equipment 40 comprises an electronic control unit 42 and a wireless communication unit 44. The electronic control unit 42 is configured to determine via the wireless communication unit 44 at least one characteristic of at least one component of the drug delivery device. The wireless communication unit 44, comprises an NFC circuit, for example, e.g. an active NFC. The electronic control 42 unit comprises a processor unit configured to execute machine-readable code (for example an app) stored in memory of the electronic control unit 42.

The electronic control unit 42 is configured to determine a proximity of the holder 16 or second module to the user equipment 40. The electronic control unit 42 is configured to determine a proximity of the drive mechanism 12 or first module to the equipment. The electronic control unit 42 is configured to determine a proximity of the respective component (holder 16, drive mechanism 12) by sending a request via the wireless communication unit 42 (e.g. the NFC circuit of the communication unit 42) to the wireless communication circuits 20, 22 of the drug delivery device 10. The respective component is considered to be or evaluated to be in proximity of the user equipment 40 when a response is received from the wireless communication circuit 20, 22 of that component. NFC is a short range communication and may require close proximity of the communication partner, e.g. less 2 cm, less than 1 cm, e.g. less than 0.5 cm. The NFC functionality may be built in standard smart watches, e.g. for a payment functionality, and, hence, is readily available for proximity detection.

The first and second wireless communication circuit 20, 22 each may be configured to respond to a request (from the user equipment) by sending a response comprising a device-type identifier. The responses from the wireless communication circuits 20, 22 of the drug delivery device 10 may each include a component-type identifier, e.g. one for the first module and one for the second module.

The electronic control unit 42 is configured to determine from the received type identifiers whether the components are compatible. This may be performed via a look-up table stored in the user equipment 40, which links compatible component-type identifiers with one another. In case an incompatibility is detected this may be communicated to the user via the user equipment.

The electronic control unit 42 is configured to send via the communication unit 44 an unlock command to the electronic system 14 of the drug delivery device 10 to unlock at least one usability function of the drug delivery device 10. The electronic control unit 42 is configured to send the unlock command when the components (holder 16 or second module and drive mechanism 12 or first module) are determined to be compatible with each other and/or when they are determined to be in proximity to the user equipment 40. The unlock command is preferably sent via a Bluetooth communication circuit, e.g. a Bluetooth Low Energy circuit, included in the wireless communication unit 44, for example.

The electronic system 14 of or for the drug delivery device comprises a system wireless communication unit or further wireless communication unit 30 configured to wirelessly receive an unlock command from the user equipment 40. The further wireless communication unit 30 comprises a Bluetooth Low Energy communication circuit for receiving the unlock command and/or communicating with the user equipment. The electronic system 14 comprises a system electronic control unit or further electronic control unit 32 configured to, when the unlock command is received, activate one or more usability functions of the drug delivery device, e.g. two usability functions. For the respective activation, activation commands may be issued by the control unit 32. The first and second wireless communication circuits 20, 22 are expediently electronically independent from the electronic system 14. The electronic system 14 further comprises a power source, e.g. a battery such as one or more coin cells to power the respective elements of the electronic system (not explicitly shown). The electronic control unit 12 may be or may comprises an ASIC or a microcontroller or another, e.g. programmable, processor. The elements of the electronic system, e.g. the communication unit 30 and the control unit 32 may be arranged on a common conductor carrier, e.g. a circuit board such as a flexible circuit board.

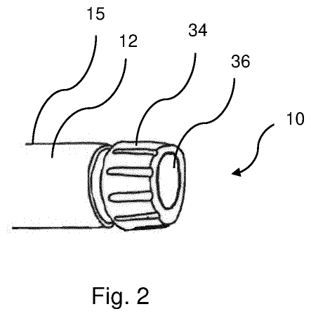

The drive mechanism 12 comprises a dose setting member 34. The dose (setting) member 34 is a turn dial or knob. The dose setting member 34 forms a proximal end of the drug delivery device 10. The drive mechanism 12 comprises a locking mechanism (not explicitly shown) to keep the dose setting member 34 in position relative to the housing 15 in a locked state. The locking mechanism may comprise a locking feature, e.g. a pin or bar to keep the dose setting member 34 in a locked state. The further electronic control unit 32 is configured to power an activation circuit to release the dose setting member 34 from the locked state, e.g. by issuing an activation command, expediently only after the unlock command has been received. Releasing the dose setting member 34 from the locked state causes the dose setting member 34 to perform an emerging motion or proximal motion. The member 34 can “pop” out or move from a locked position (see FIG. 1) in a proximal direction relative to housing 15 into an operating position (see FIG. 2). In the locked position, the dose setting member 34 may be decoupled from the drive mechanism and/or a dose setting movement may be prevented. Alternatively or additionally, in the locked position, the dose setting member is rotationally and/or axially locked relative to the housing 15. In the operating position, movement of the dose setting member for the dose setting operation is expediently allowed, e.g. involving rotational and proximal movement relative to the housing 15. The axial distance between the operation position and the non-operating or locked position may be less than 1 cm, e.g. less than 0.5 cm. The electronic system 14 is expediently integrated into the dose setting member 34. The electronic system moves proximally together with the dose setting member during the dose setting operation.

The activation circuit may for example be configured to produce a magnetic field to release a magnetic locking mechanism. The drive mechanism 12 may for example include a spring to cause the emerging motion of the dose setting member 34 as soon as the lock is released, e.g. by moving the locking feature. Other (un) locking mechanisms, e.g. heat based or electrical field based or unlocking a lock via relative movement induced by the electronic control unit or a motor, are also possible.

The drive mechanism 12 further includes a dose delivery activation member 36. The dose delivery activation member 36 is arranged on a proximal end of the dose setting member 34. The dose setting member and the dose delivery activation member may be integrated into a common dose member. The dose delivery activation member 36 or dose member is a button. Alternatively, when two separate members are provided, the dose delivery activation member 36 can be moved in relation to the dose setting member 34 when pressed for activating or initiating the dose delivery operation.

When the dose setting member 34 is unlocked, the dose setting member 34 may be rotated and moved (e.g. in a helical fashion) proximally relative to the housing 15, i.e. from the operating position (the position after the member 34 has been unlocked) to a dose set position. The distance of the movement of the dose setting member 34 is proportional, e.g. directly proportional, to a set dose amount. When the dose delivery activation member 36 is pressed, the dose setting member 34 is expediently mechanically coupled with a transmission unit of the drive mechanism 12 and pressure exerted on the dose delivery activation member 36 and, preferably, therewith upon the dose setting member 34 by the user is converted into movement of a piston rod of the drive mechanism (not explicitly shown). The piston rod acts on the medicament container 19, e.g. on the bung in the cartridge, to cause medicament to be ejected from the drug delivery device 10. The force acting upon the dose setting member 34 causes the dose setting member to move back into its original position. When the dose setting member 34 reaches a distal end position, e.g. a position close (st) to the housing 15, the dose setting member 34 may be locked again. Thus, the end position may correspond to the locked position relative to the housing 15. The lock may be released for the next dose setting operation, preferably via the user equipment. Alternatively or additionally to the dose setting member 34, the dose delivery activation member 36 may be held in a locked state until the activation circuit or a further activation circuit is powered, e.g. if the members are not integrated into one dose member.

The drug delivery device 10 further comprises a dose display 38. When the drug delivery device 10 is in a locked state the dose display 38 is blank. The dose display 38 is arranged on a side of the housing 15. When the unlock command is received, the further electronic control unit 32 is configured to unblank the dose display 38, e.g. by powering an (other) activation circuit. The dose display 38 may be unblanked by removing an obstruction, e.g. a shutter element, from a viewing window in the region of the dose display. This movement may be spring driven or motor-driven. When unblanked, a number sleeve with dose indicia indicating the size of the currently set dose may be visible or revealed in the window. Alternatively, an electronic dose display may be switched on by the activation circuit or the number sleeve may be displaced relative to the window, e.g. along with the dose (setting) member.

The user equipment 40 further comprises a signaling unit 46, wherein the electronic control unit 42 is configured to signal to the user via the signaling unit 46 a ready state after sending the unlock command. The signaling unit 46 comprises a display, e.g. a dot-matrix display (e.g. liquid crystal display or organic light emitting diode display). The electronic system 14 comprises a system signaling unit or further signaling unit 39, wherein the further electronic control unit 32 is configured to signal a ready state to the user via the further signaling unit 39 when the usability function is active or has been activated. The further signaling unit 39 comprises a plurality of, e.g. three light emitting diodes of different colors (e.g. red, green and blue) and is able to display a range of different colors, e.g. at least yellow and green. The further signaling unit 39 is integrated into the dose member 34/36, e.g. in the proximal end surface of the dose delivery activation member 36. The dose delivery activation member 36 may have an at least partially transparent or at least translucent surface to have light from the signaling unit 39 that may be arranged behind the dose delivery activation member 36 shine though the surface when activated.

A method for controlling a before mentioned drug delivery device 10 or preparing the device for a dosing operation, e.g. by a before mentioned user equipment 40, may comprise a setup portion or step.

During the setup portion, the user equipment 40 and the electronic system 14 of the drug delivery device 10 are paired. This may be done in various ways. For example, the user equipment can request a device ID from the drug delivery device and store it in memory. During the setup portion a medicament administration schedule can be set and/or timers or clocks of the user equipment 40 and the electronic system 14 may be synchronized. An alarm for an upcoming injection (medicament administration) can be set.

When the next injection is due, the method enters the alarm portion or step. During the alarm portion the user equipment 40 alarms the user. The electronic control unit 42 is configured to use the signaling unit 44 to provide the alarm. Alternatively or additionally, during the alarm portion the drug delivery device 10 gives an alarm. The further electronic control unit 32 is configured to signal the alarm via the further signaling unit 39, e.g. an LED. The further electronic control unit 32 may be configured to signal the alarm by having the signaling unit emit a constant or flashing signal of a specific alarm color (e.g. yellow), for example. The alarm color may be set by the user and/or the user equipment 40 during the setup portion. The alarms on the device and on the equipment may be correlated, e.g. of the same color, the same symbols etc. In this way, the alarm may distinguish a particular device, such as from other devices used in the same environment, e.g. the same household. Depending on the capabilities of the signaling unit other types of alarms than an alarm color, e.g. an audible alarm, an alarm symbol or an alarm text, or combinations of different alarms may be applied. Alternatively and/or additionally to the LEDs, the further signaling unit may comprise a dot-matrix display and/or a display foil. The (dot-matrix) display may be used to display a symbol as an identification mark, e.g. instead of the alarm color. The electronic control unit 42 of the user equipment may be configured to display the symbol via the signaling unit 46. During the alarm portion, the electronic control unit 42 is configured to display the alarm color as an identifying mark of the drug delivery device 10 to be used for the injection via the signaling unit 46, e.g. together with an audible alarm (e.g. emitted through a speaker).

During the alarm portion or thereafter the proximity of the user equipment 40 to component(s) of the drug delivery device can be checked from time to time (e.g. regularly and/or periodically). The electronic control unit 42 is configured to send out periodic requests via the wireless communication unit 44 (e.g. via the NFC-communication circuit).

During the alarm portion or thereafter, the method includes wirelessly determining at least one characteristic of at least one component of the drug delivery device 10 by the user equipment.

When the alarm portion is completed or even without a preceding alarm portion, the method may involve the determination of at least one characteristic of the component(s) of the device in a preparing portion or step of the method. In this step, the device may be prepared for a dosing operation.

When the drug delivery device 10 enters proximity (e.g. a few/couple of centimeters or less than 0.5 cm, see above) of the user equipment 40, the wireless communication circuits 20, 22 of the component(s) of the drug delivery device 10 receive the requests and send signals in response thereto. Therefore, proximity of the user equipment 10 and the components is determined wirelessly as the wireless communication unit 44 receives the responses from the wireless communication circuits 20, 22 of the components of the drug delivery device 10. Before user equipment and component(s) have been brought into proximity, the equipment may also send requests but without an according response, e.g. due to the limited range of the communication circuits 20, 22. Confirming proximity between the component(s) of the device and the user equipment may require a distance between the component(s) and the wireless communication unit 44 which is less than the distance between the device when held in the user's hand and the user equipment worn on the user's wrist adjoining that hand. This means that the device may have to be brought in very close proximity to the user equipment for confirming proximity. We note that it is also possible that proximity is confirmed when the user holds the device or the component(s) in his hand.

Alternatively to passive circuits, the respective wireless communication circuit 20 or 22 can be an active circuit, e.g. an NFC-circuit and/or powered by a, preferably printable, battery. The circuits 20, 22 may be configured to send signals during the alarm portion without having received a prior request. In this case, the electronic control 42 unit may be configured to not send requests via the wireless communication unit 44 (at least not via the NFC communication circuit or not at all). Rather it may wait for a signal received from the circuit(s) to confirm proximity between the device or its component(s) and the user equipment.

The signals received from the wireless communication circuits 20, 22 in the wireless communication unit 44 comprise component-type identifiers. The electronic control unit 42 checks the received component-type identifiers for an identifier identifying a (dedicated) container module. The electronic control unit 42 checks the received component-type identifiers for an identifier identifying a (dedicated) drive mechanism suitable for the (dedicated) container module. The electronic control unit 42 checks whether the received component type identifiers belong to compatible components, e.g. to a container module which has been dedicated to a drive mechanism. We note that the compatibility check may be independent of the proximity check. Hence, the disclosure may relate to either one of these checks being performed or the checks being performed in combination.

Information on the compatibility of the modules is expediently indicated to the user via the signaling unit 46, e.g. textual information and/or color-based information. For example, if the components are found to be incompatible, a red symbol may be displayed by the signaling unit 46 of the user equipment. This may prompt the user to exchange at least one of the components, e.g. to use another container module with the mechanism module. If the components are found to be compatible, the indicated information may reassure the user that the device is properly set up. Alternatively or additionally, it may be indicated whether the device under investigation is in the proximity of the user equipment.

The electronic control unit 42 may further, e.g. alternatively or additionally to the check(s) discussed above, communicate via the wireless communication unit 44 and the further wireless communication unit 30 with the further electronic control unit 32 to request information about a due time of injection and/or a device identifier from the device, e.g. to verify that the drug delivery device 10 in proximity is the correct drug delivery device.

When the components of the drug delivery device 10 are determined to be in proximity, determined to be compatible (have the correct pairing) and/or the drug delivery device has determined to be the correct one, the electronic control unit 42 sends an unlock command to the drug delivery device 10 for unlocking the usability function(s) of the drug delivery device. As a consequence, the dose setting member 34 is released from its locked state and the dose display 38 is unblanked or revealed, e.g. by moving a shutter member away from the window to reveal the number sleeve. We note that either revealing the dose display or number sleeve or releasing the dose setting member without the other one is also covered by the present disclosure.

After the unlock command was sent, the method enters the dose setting portion or step. In the dose setting portion, the ready state is signaled or indicated to the user. The further signaling unit 39 may change a color and/or a signaling mode. E.g. the dose delivery activation member may be illuminated green, and may be flashing. Other indications are possible, of course.

After the unlock command has been sent—e.g. in response to the dose setting member reaching its operating position (in this case the further electronic control unit of the electronic system 14 can instruct an according signal to be transmitted from the electronic system to the user equipment 40) or after a predetermined time, e.g. less than 1 s or less than 2 s, has elapsed after the unlock command has been sent—the user equipment 40 preferably indicates to the user a dose amount to be set in the drug delivery device 10, e.g. on the signaling unit of the user equipment. The user can then set the indicated dose, e.g. by rotating the dose setting member until the corresponding dose amount appears in the dose display 38. During dose setting, the dose amount displayed in the dose display changes to always reflect the currently set dose. The length of the device increases in proportion to the dose amount. The higher the dose amount, the longer the device. The dose setting member is moved proximally relative to the housing 15 in the course of dose setting. The set dose can then be delivered, e.g. by the user moving the dose delivery activation member 36 distally towards the housing 15, e.g. until the member is locked again axially to the housing in its end position. The size of the delivered dose may be determined in the electronic system 14 and stored therein and/or be transmitted to the user equipment e.g. along with a timestamp.

It will be appreciated that the present disclosure covers also alternatives to the implementation in the exemplary embodiment described above. For example: According to alternatives, the drive mechanism and the holder comprise different types of communication circuits. For the proximity detection in the above described example, near field or short range communication is used. Specifically presence of the circuits within the communication range is used to confirm proximity. It is also possible to use non-near-field or longer range wireless communication, e.g. Bluetooth such as Bluetooth low energy (BLE) for distance determination. As opposed to NFC, non-near-field communication may be more suitable to detect whether the device is in the user's hand as the separation between the wrist and the hand may be out of the communication range for NFC circuits. For this purpose, proximity or distance detection or measurement may be used, e.g. applying:
BLE with signal strength detection (RSSI),
BLE with time of flight measurements,
Ultra-wide band (UWB) distance measurement,
modulated magnetic fields (e.g. detectable by the magnetormeter or compass of the user equipment or smart watch.

The range of NFC may be too small to detect, whether the device is in the user's hand and, hence, preparing the device for the dosing operation may require the device being moved closer to the user equipment to perform the one or more of the checks required before the respective usability function is unlocked. UWB, e.g. pursuant to the standard IEEE 802.15.4-2011, may be particularly suitably to be used for distance measurements in distances exceeding the range of NFC and/or for distances typical between the wrist and a device held in the hand adjacent the wrist. The drive mechanism may not comprise an independent communication circuit but one included into the electronic system, particularly if the system is integrated into the mechanism module. The user equipment then is configured to determine the characteristics of the mechanism module by sending a request to and/or receiving signals from the further wireless communication unit. Also, we note that the summary section may describe features which are not expressly described in the description of the exemplary embodiment but do nevertheless apply for the embodiment as described in the summary section. Hence, the summary section is incorporated by reference into the description of the embodiments.

The scope of protection is not limited to the examples given herein above. Any invention disclosed herein is embodied in each novel characteristic and each combination of characteristics, which particularly includes every combination of any features which are stated in the claims, even if this feature or this combination of features is not explicitly stated in the claims or in the examples.

### REFERENCE NUMERALS

10 drug delivery device
12 drive mechanism
14 electronic system
16 cartridge holder
18 receptacle
19 medicament container
20 wireless communication circuit
22 wireless communication circuit
30 further wireless communication unit
32 further electronic control unit
34 dose setting member
36 dose delivery activation member
38 dose display
39 further signaling unit
40 user equipment
42 electronic control unit
44 wireless communication unit
46 signaling unit

1. A user equipment, e.g. a smart watch, comprising:
an electronic control unit (42), and
a wireless communication unit (44) operatively connected to the electronic control unit, the electronic control unit (42) being configured
to control operation of the wireless communication unit (44), the wireless communication unit being operable to determine at least one characteristic associated with at least one component of a drug delivery device (10),
to evaluate the at least one determined characteristic, and
based on the result of the evaluation to issue or to decide whether to issue one or more control unit commands, wherein the one or more control unit commands comprise commands configured to cause
a) an equipment signaling unit of the user equipment to present information relating to the drug delivery device to the user, the information being based on the at least one determined characteristic, and/or to cause
b) the wireless communication unit to send a system command to an electronic system of or for the drug delivery device.

2. The user equipment of claim 1,
wherein the electronic control unit is configured to decide, based on the result of the evaluation, whether the wireless communication unit (44) should send at least one unlock command as the system command to the electronic system (14) to unlock at least one usability function of the drug delivery device (10), and wherein the user equipment is configured to send the unlock command only when the result of the evaluation is that the at least one determined characteristic meets at least one predetermined criterion.

3. The user equipment of claim 1 or 2,
wherein the user equipment (40) is a wearable device, e.g. a smart watch.

4. The user equipment of any one of the preceding claims,
wherein the at least one determined characteristic comprises the distance between or the proximity of the component of the drug delivery device (10) and the user equipment and evaluating the at least one determined characteristic comprises determining whether the component or the entire drug delivery device is within a predetermined distance of the user equipment, and wherein, only when the component or the drug delivery device is within the predetermined distance, the unlock command is sent as the system command.

5. The user equipment of claim 4,
wherein the user equipment is configured to use UWB (ultra-wide band) distance measurement to determine the distance between or the proximity of drug delivery device and user equipment.

6. The user equipment of any one of the preceding claims,
wherein the at least one determined characteristic comprises identifiers of at least two components of the drug delivery device (10), e.g. component-type identifiers, wherein evaluating the at least one determined characteristic comprises determining whether the at least two components of the drug delivery device are compatible with one another, and wherein the user equipment is configured to send the unlock command only when the components are compatible with one another.

7. The user equipment of claim 6,
wherein the user equipment is configured to determine a first identifier of a first component and a second identifier of a second component of the device in one relative position with respect to the drug delivery device, e.g. simultaneously.

8. The user equipment of any one of the preceding claims,
wherein the user equipment (40) comprises the equipment signaling unit (46), and wherein the equipment signaling unit is operatively connected to the electronic control unit (42) and, on instruction by the electronic control unit (42), configured to indicate at least one of the following:
a device identification to indicate a drug delivery device (10) which the user should use for an upcoming medicament administration,
information on the compatibility of components of the drug delivery device,
a ready state of the drug delivery device, especially a state after having sent the unlock command, and/or
a dose amount to be set in the drug delivery device (10) for a dose delivery operation.

9. An electronic system (14) for a drug delivery device (10), wherein the electronic system comprises a system wireless communication unit (30) configured to wirelessly receive an unlock command from a user equipment (40), e.g. a user equipment according to any of claims 1 to 8, and a system electronic control unit (32) which is operatively connected to the system wireless communication unit and configured to, in response to the unlock command, issue an activation command to activate at least one usability function of the drug delivery device (10).

10. The electronic system of claim 9,
wherein activating the at least one usability function comprises switching on or revealing a dose display (38) of the drug delivery device.

11. The electronic system of claim 9 or 10,
wherein activating the at least one usability function comprises unlocking a dose member (34, 36) of the drug delivery device (10), wherein the drug delivery device is configured such that the dose member, in response to the activation command, is moved relative to a housing of the drug delivery device, e.g. axially, from a locked or non-operating position into an operating position.

12. The electronic system of any one of claims 8 to 10,
wherein the system electronic control unit (32) is configured to issue a ready command to cause a system signaling unit (39), in response to the ready command, to indicate to the user that the drug delivery device is ready for commencing a dosing operation.

13. A drug delivery device, comprising
a container module (16) as a component of the drug delivery device, the container module comprising or being configured to retain a container with drug, and
a mechanism module (12) as a component of the drug delivery device, the mechanism module comprising a drive mechanism for setting a dose of medicament to be delivered from the container and delivering the set dose,
wherein the container module and the mechanism module are releasably connectable to one another, wherein the container module comprises an identifier for being communicated to the wireless communication unit of the user equipment according to any one of claims 1 to 8, and wherein
the mechanism module comprises an identifier for being communicated to the wireless communication unit of the user equipment according to any one of claims 1 to 8.

14. The drug delivery device of claim 13,
further comprising the electronic system (14) of any one of claims 7 to 10 as a component of the drug delivery device.

15. A method for preparing a drug delivery device (10) for a dosing operation, e.g. a drug delivery device according to claim 13 or 14, with the aid of a user equipment (40), e.g. a user equipment according to claims 1 to 8, the method comprising the following steps:
wirelessly determining at least one characteristic of at least one component of the drug delivery device (10) via the user equipment (40),
unlocking at least one usability function of the drug delivery device (10) when the at least one determined characteristic meets a predefined criterion.

16. A machine-readable code that when loaded on and/or executed by an electronic control unit of a user equipment (40) causes the user equipment to function as a user equipment according to any of claims 1 to 8 or controls the method of claim 15.

17. A data storage medium comprising the machine-readable code of claim 16.

18. A set comprising:
one of, an arbitrarily selected plurality of or all of the following:
the user equipment (40) of one of claims 1 to 8,
the electronic system (14) of one claims 9 to 12,
one or more components of the drug delivery device (10) of claims 13 to 14,
the drug delivery device (10) of claims 13 to 14,
the machine-readable code of claim 16, and
the data storage medium of claim 17.

19. A user equipment, e.g. a smart watch, comprising:
an electronic control unit (42), and
a wireless communication unit (44) operatively connected to the electronic control unit, the electronic control unit (42) being configured
to control operation of the wireless communication unit (44), the wireless communication unit being operable to determine at least one characteristic associated with at least one component of a drug delivery device (10),
wherein the at least one characteristic comprises the distance between or the proximity of the component and the user equipment, e.g. its wireless communication unit,
to evaluate the at least one determined characteristic, and
based on the result of the evaluation to issue or to decide whether to issue one or more control unit commands, wherein the one or more control unit commands comprise commands configured to cause
a) an equipment signaling unit of the user equipment to present information relating to the drug delivery device to the user, the information being based on the at least one determined characteristic, and/or to cause
b) the wireless communication unit to send a system command to an electronic system of or for the drug delivery device.
